(19) United States
(12) Patent Application Publication
(10) Pub. No.: US 2025/0277789 A1
(43) Pub. Date: Sep. 4, 2025

(54) METHOD OF IMPROVING PREDICTION ACCURACY AND REDUCING FALSE NEGATIVE RATE OF SEVERE DENGUE PREDICTION IN SUBJECT

(71) Applicant: NATIONAL CHENG KUNG UNIVERSITY, Tainan City (TW)

(72) Inventors: Tzong-Shiann HO, Tainan City (TW); Ya-Lan LIN, Tainan City (TW); Hong-Jyun HUANG, Tainan City (TW); Yung-Chun CHUANG, Tainan City (TW); Yu-Wei CHENG, Tainan City (TW)

(21) Appl. No.: 19/203,213

(22) Filed: May 9, 2025

Related U.S. Application Data
(63) Continuation-in-part of application No. 17/533,535, filed on Nov. 23, 2021.

(30) Foreign Application Priority Data
Feb. 23, 2021 (TW) 110106367

(51) Int. Cl. G01N 33/569; G01N 33/68

(52) U.S. Cl. CPC G01N 33/56983; G01N 33/6854; G01N 2333/185; G01N 2469/20

(57) ABSTRACT

The present invention relates to a method of improving prediction accuracy of severe dengue prediction in a subject. In the method, a non-structural protein 1 (NS1) and content ratio of endogenous anti-NS1 antibodies of dengue virus in an ex vivo biological specimen are detected and crossly compared, leading in reduce of false negative rates as well as improved prediction accuracy of patients with severe dengue.

### CROSS-REFERENCE TO RELATED APPLICATION

The present application is a continuation-in-part of the application Ser. No. 17/533,535, filed on Nov. 23, 2021, and claims priority under 35 U.S.C. 119(a) to Taiwan application serial number 110106367, filed on Feb. 23, 2021, which incorporated herein by reference in their entireties.

### BACKGROUND

### Field of Invention

The present invention relates to a medical examination method. More specifically, the present invention relates to a method of improving prediction accuracy of severe dengue prediction in a subject by detecting a non-structural protein 1 (NS1) and a content ratio of endogenous anti-NS1 antibodies of dengue virus in an ex vivo biological specimen.

### DESCRIPTION OF RELATED ART

Dengue fever is a disease that quickly spreads and has a short course, and about 390 million people are infected by dengue virus worldwide every year. In the past 20 years, Taiwan has experienced several regional dengue fever epidemics. In addition to the epidemic in Southern Taiwan, there have been clustered outbreaks of indigenous dengue cases in New Taipei City and Taichung City that are not the main affected areas, which shows that dengue fever has a trend of localization, and its threat spreads to the whole Taiwan. Dengue fever has become an important emerging infection and public health problem in Taiwan.

The clinical symptoms of dengue patients vary greatly, from fever like a common cold to fatal dengue shock syndrome or dengue hemorrhagic fever. Early diagnosis of severe dengue fever can provide timely disease monitoring and management. However, current test techniques yet cannot meet the clinical need to predict the severity of dengue fever. New research in recent years has further found that NS1 is an important viral toxin, which is known to cause important pathogenic effects such as plasma leakage, dysfunction of blood coagulation, and thrombocytopenia during severe dengue. In addition, an anti-NS1 antibody has been confirmed in animal experiments to have the effect of treating hemorrhagic lesions caused by dengue infection.

It has been found in previous studies that, the viral toxin NS1 forms a complex with thrombin or prothrombin in serum samples from patients with dengue infection, prolonging activation of partial thromboplastin and thus causing severe bleeding. Therefore, the current test process involves first collection and testing (briefly referred to as first collection) for suspected dengue infection cases and fast screening for NS1 antigen of the dengue virus. If the fast screening result in the first collection is negative, the process further involves second collection and testing (briefly referred to as second collection) and dengue virus-specific real-time PCR, RT-PCR, and IgM/IgG tests, where the IgM/IgG test refers to seroconversion of anti-dengue IgM or IgG antibodies or at least four-fold increase of a positive result of IgG antibodies in the serum of the second collection.

However, the current test process has a high false negative rate for predicting severe dengue in patients, i.e., the probability to predict severe dengue as non-severe dengue in patients is high, leading to a misjudgment and those patients in need may not get care right away. In view of this, it is in urgent need to provide a method of improving prediction accuracy of severe dengue prediction in a subject, so as to solve the conventional problem of a high false negative rate of testing results of dengue fever patients.

### SUMMARY

Accordingly, an aspect of the present invention provides a method of improving prediction accuracy of severe dengue prediction in a subject, in which a non-structural protein 1 (NS1) and endogenous anti-NS1 antibodies of dengue virus in an ex vivo biological specimen are detected and crossly compared, leading in reduction of false negative rates as well as improved prediction accuracy of severe dengue prediction in a subject.

According to the foregoing aspect of the present invention, a method of improving prediction accuracy of severe dengue prediction in a subject is provided. For example, the method includes: providing an ex vivo biological specimen; performing two detection steps on the ex vivo biological specimen, so as to obtain a first test result and a second test result; and diagnosing the subject with severe dengue when the first test result is higher than a first cut-off value or the second test result is lower than a second cut-off value.

In the aforementioned embodiment, the ex vivo biological specimen has not been diagnosed or differentially diagnosed with a dengue virus infection or a suspected dengue virus infection.

In the aforementioned embodiment, the first test result includes a concentration of NS1, and the second test result includes a content ratio of a first antibody and a second antibody, the first antibody specifically recognizes the 109^{th} to the 122^{nd} amino acid residues of the NS1, and the second antibody specifically recognizes the 114^{th} to the 119^{th} amino acid residues.

In the aforementioned embodiment, when the first test result is higher than a first cut-off value and the second test result is lower than a second cut-off value, the subject corresponding to the ex vivo biological specimen is predicted as severe dengue, and a false negative rate of the severe dengue prediction is lower than 5%.

In the aforementioned embodiment, the ex vivo biological specimen includes blood, urine, saliva, tissue fluid and/or lymphatic fluid.

In the aforementioned embodiment, the first cut-off value is 660 ng/ml to 670 ng/ml. In the aforementioned embodiment, the second cut-off value is 0.4294.

In the aforementioned embodiment, the serotypes of the dengue virus include type 1, type 2, type 3 and type 4.

In the aforementioned embodiment, the first antibody and the second antibody are IgG and/or IgM.

In the aforementioned embodiment, the subject can be a mammal, for example, a human being.

In the aforementioned embodiment, the true positive rate of the severe dengue prediction is at least 95%.

In the aforementioned embodiment, the two detection steps are performed by immunoassays including an enzyme-linked immunosorbent assay (ELISA), western blot assay, lateral laminar flow immunoassay, multiple immunoassay, radio immunoassay, immunoradiometric assay, fluorescence immunoassay, chemiluminescence immunoassay and/or immunoturbidimetry.

### BRIEF DESCRIPTION OF THE DRAWINGS

The disclosure can be more fully understood by reading the following detailed description of the embodiment, with reference made to the accompanying drawings as follows.

FIG. 1 shows a scatter plot 110 illustrating a content ratio of modified NS1-WD IgG/NS1 IgG in sera of various dengue fever patients according to an embodiment of the present invention.

FIG. 2 shows a Receiver operating characteristic (ROC) curve in a severe dengue prediction with a conventional method.

FIG. 3 shows a ROC curve in a severe dengue prediction in terms of NS1 antigens in the sera of dengue fever patients according to an embodiment of the present invention.

FIG. 4 shows a ROC curve in a severe dengue prediction in terms of a content ratio of the endogenous anti-NS1 antibody (i.e., anti-NS1-WD peptide antibody/anti-NS1 peptide antibody) in the sera of dengue fever patients according to an embodiment of the present invention.

FIG. 5 shows a ROC curve in a severe dengue prediction in terms of a content ratio of the endogenous anti-NS1 antibodies in the sera of dengue fever patients by means of mouse antibody detection according to a comparative example.

### DETAILED DESCRIPTION

Reference will now be made in detail to the present embodiments of the invention, examples of which are illustrated in the accompanying drawings. Wherever possible, the same reference numbers are used in the drawings and the description to refer to the same or like parts.

All documents cited herein are deemed to be specifically and individually incorporated into references through citation of each individual document or patent application. Where a definition or use of a term in an incorporated reference is inconsistent or contrary to the definition of that term provided herein, the definition of that term provided herein applies and the definition of that term in the reference does not apply.

To explain the specification, the following definitions are applicable. Unless inappropriate in the context, the terms “(a/an)” and “(the/said)” mentioned herein are defined as “one or more” and include plural forms. Additional definitions are set forth throughout the detailed description.

As described above, the present invention provides a method of improving prediction accuracy of severe dengue prediction in a subject, in which a non-structural protein 1 (NS1) and endogenous anti-NS1 antibodies of dengue virus in an ex vivo biological specimen are detected and crossly compared, leading in reduction of false negative rates of testing results as well as improved prediction accuracy of severe dengue prediction in a subject.

The term “dengue virus” mentioned herein can be used alternately with “dengue fever virus” and “DE NV”. The serotypes of the dengue virus can include but are not limited to type 1, type 2, type 3 and type 4.

In serum samples from patients with dengue infection, NS1 of the dengue virus forms a complex with thrombin or prothrombin, prolonging activation of partial thromboplastin and thus causing severe bleeding. In the prior art, it is determined whether a subject is infected with dengue virus by detecting whether there is a complex of NS1 and thrombin or a complex of NS1 and prothrombin in an in vitro biological sample. However, among patients with dengue infection, different patients vary greatly in the severity of the disease, and a severe disease can even lead to death. The current test process merely tests the complex of NS1 and thrombin or the complex of NS1 and prothrombin, and the test results have a high false negative rate, thus failing to accurately determine the severity of the disease. As a result, it is likely to neglect the disease condition and delay the treatment.

Therefore, the method of the present invention performs two detection steps for an ex vivo biological specimen, and crossly compares a first test result and a second test result, so as to determine whether the ex vivo biological specimen contains NS1 of a high concentration or a low content ratio of two endogenous anti-NS1 antibodies of dengue virus, leading in reduction of false negative rates as well as improved prediction accuracy of severe dengue prediction in a subject. It is noted that the terms “test” and “detect” (or examine) mentioned herein can be alternately used, and the terms “true positive rate”, “accuracy” and “accuracy rate” can also be alternately used.

The “ex vivo biological specimen” generally refers to a range of influence within a subject with dengue virus infection and is not particularly limited, which can include but are not limited to blood (e.g., serum, plasma, or whole blood), urine, saliva, lymphatic fluid, or tissue fluid; or nearby tissues or cells through which the blood, urine, lymphatic fluid, or tissue fluid flows. In some embodiments, the ex vivo biological specimen preferably contains cells infected with the dengue virus. The cells can include but are not limited to nerve cells, muscle cells, liver cells, endothelial cells, blood cells, and lymphocytes; and preferably include endothelial cells or blood cells of mammals. In other embodiments, the ex vivo biological specimen can be, for example, a fresh, tissue-cultured, or refrigerated or frozen sample. In some specific examples, the ex vivo biological specimen can be subjected to conventional pretreatment (for example, purification, centrifugation, extraction, or concentration), so as to increase the concentration of a substance (for example, the NS1, or the endogenous anti-NS1 antibodies) to be detected. In some embodiments, the ex vivo biological specimen has not been diagnosed or differentially diagnosed with a dengue virus infection or a suspected dengue virus infection. The term “prediction”, and “predict” herein refers to differentiate, classify, group or differentially diagnose the subject having severe dengue or non-severe dengue with the biological specimen before the subject has been diagnosed or differentially diagnosed with other methods.

The “first test result” refers to a concentration of NS1 antigen. Specifically, the first test result is obtained by detecting the concentration of NS1 with an antibody, where the antibody is an exogenous anti-NS1 antibody.

The “exogenous antibody” can be, for example, a monoclonal antibody or a polyclonal antibody. In some embodiments, the antibody that specifically recognizes the NS1 can be, for example, a monoclonal antibody. In other embodiments, the antibody that specifically recognizes the NS1 complex can be, for example, a polyclonal antibody.

The exogenous antibody includes an antibody-based binding moiety, or immunoglobulin molecules and their immunologically active determinants, for example, molecules containing an antigen-binding site that binds immune-specifically to the NS1. The type of exogenous antibody can include but are not limited to IgG, IgA, IgM, IgE, or the like, instead of limitation.

The exogenous antibody can also include an antigen-binding fragment that specifically reacts with the NS1. The antigen-binding fragment is not limited in structure, and in consideration of the structural stability of the complementarity-determining region (CDR), can have a complete antibody structure or simplified antibody structure, such as a single-chain variable fragment (scFv), scFv dimer [(scFv)₂], scFv trimer [(scFv)₃], a variable fragment (Fv), a Fab fragment, a Fab′ fragment, a F(ab′)₂ fragment, a nanobody (also referred to as a single domain antibody (sdAb) or a heavy-chain antibody), or any combination of the above.

After a patient is infected with the dengue virus, in addition to the NS1 that is detected in the ex vivo biological specimen, an anti-NS1 “endogenous antibody” is also produced in the body. The inventors also find that a higher concentration of the NS1 and a lower content ratio of the endogenous antibodies against two different specific NS1 peptide sequences in the body of the patient are relevant to the severity of the dengue disease. In an embodiment, the concentrations of the endogenous anti-NS1 antibodies can be detected in the ex vivo biological specimen to obtain the quotient, so as to obtain a second test result.

In this embodiment, the endogenous anti-NS1 antibodies include a first antibody and a second antibody. In some instances, the first antibody refers to an endogenous antibody that specifically recognizes the 109^{th} to 122^{nd} amino acid residues of the NS1 of the dengue virus, where the 109^{th} to 122^{nd} amino acid residues of the NS1 can be defined as modified NS1-WD peptide, and that is referred hereafter to as an antibody against modified NS1-WD peptide or an anti-NS1-WD peptide antibody. It is found in past clinical studies that the higher the concentration of such an anti-NS1-WD peptide antibody in the body, the less likely it is for the patient to develop a severe disease. In addition, the quality and quantity of the anti-NS1-WD peptide antibody are also relevant to the severity of the disease.

In some other instances, the second antibody refers to an endogenous antibody that specifically recognizes the 114^{th} to 119^{th} amino acid residues of the NS1 of the dengue virus, where the 114^{th} to 119^{th} amino acid residues of the NS1 belong to a conserved sequence of four serotypes of the dengue virus and facilitate recognition of NS1 of the four serotypes of the dengue virus, and that is also referred to as an antibody against NS1 of all serotypes or an anti-NS1 antibody.

In other embodiments, the isotype of the endogenous anti-NS1 antibody is not limited and can be, for example, IgG and/or IgM. In some specific examples, the second test result refers to a content ratio of the first antibody to the second antibody. Moreover, the lower the content ratio of the first antibody to the second antibody, the patient with a dengue virus infection is more likely to be severe dengue.

In some specific examples, a humanized antibody (hAb) can be used to specifically recognize the endogenous anti-NS1 antibody. A method for producing the hAb belongs to common knowledge in the art of the present invention. In some embodiments, the skeleton of a recipient human antibody can be used, and a CDR sequence of a rodent antibody is used to replace the corresponding sequence of the human antibody, so as to obtain a hAb, and such a hAb belongs to a human-mouse chimeric antibody. In examples of the human-mouse chimeric antibody, a human antibody germline sequence available from a public database can be selected for the skeleton of the recipient human antibody, where the ethnic group of the skeleton of the recipient human antibody is not particularly limited and depends on the ex vivo biological specimen to be detected.

The “individual”, “subject”, or “patient” mentioned herein refer to a mammal. In a specific example, the individual, subject, or patient can be, for example, a human being.

The dengue fever patients with “mild symptoms (or referred to as non-severe dengue)” mentioned herein are those who show warning signs (or referred to as warning symptoms) or have no warning signs, where the warning signs can include but are not limited to, for example, abdominal pain or tenderness, persistent vomiting, clinical fluid accumulation, mucosal bleeding, and the like. In other embodiments, mild patients who have no warning signs can be classified as group A patients, while mild patients who show warning signs can be classified as group B patients. Reference can be made to Handbook for Clinical Management of Dengue published by the World Health Organization (WHO) for relevant judgment principles.

The dengue fever patients with “severe symptoms” mentioned herein are those who show such signs as severe plasma leakage which causes shock and fluid accumulation with respiratory distress, severe bleeding, and severe organ impairment. In other embodiments, the severe patients can be classified as group C patients. Reference can be made to Handbook for Clinical Management of Dengue published by the WHO for relevant judgment principles.

The “severe dengue” mentioned herein is determined according to the method of the present invention. Generally speaking, in the medical detection field, a represents false positive or is referred to as the opposite of specificity; and β represents false negative or is referred to as the opposite of sensitivity. In application of the method of the present invention, the concentration of a non-structural protein 1 (NS1) and a content ratio of a first antibody and a second antibody against dengue virus in an ex vivo biological specimen are detected and crossly compared, and the subject for which the first test result is higher than a first cut-off value or the second test result is lower than a second cut-off value is predicted as the severe dengue, thus effectively reducing the numerical value of P (namely, reducing “the false negative rate”) and improving the true positive rate (also referred to as “prediction accuracy”) of patients with severe dengue (namely, increasing the numerical value of “1-β”). In addition, when the first test result is lower than the first cut-off value and the second test result is higher than the second cut-off value, it is determined that the subject corresponding to the ex vivo biological specimen is classified as a non-severe dengue group. The first cut-off value and the second cut-off value are obtained with ROC curves in a severe dengue prediction in terms of the concentration of NS1 antigen and the content ratio of the endogenous anti-NS1 antibodies (i.e., anti-NS1-WD peptide antibody/anti-NS1 peptide antibody), respectively. In some embodiments, the first cut-off value can be 660 ng/ml to 670 ng/ml, for example. In some embodiments, the second cut-off value can be 0.4294, for example.

In the aforementioned embodiments, the two detection step can be performed by immunoassays including but are not limited to an ELISA, western blot assay, lateral laminar flow immunoassay, multiple immunoassay, radio immunoassay, immunoradiometric assay, fluorescence immunoassay, chemiluminescence immunoassay and/or immunoturbidimetry. In other embodiments, other means can also be used to detect the NS1 and the endogenous anti-NS1 antibody in the ex vivo biological specimen.

It should be additionally noted that the conventional test processes mostly determine the severity of the dengue fever patients in order according to a single test result, failing to effectively reduce the false negative rate of the test results. In some specific examples, compared to the false negative rate of about 10% to 30% in the current test process, the method for predicting “the severe dengue” in a subject of the present invention can decrease the false negative rate of the test results to about 4.5% after diagnosis and confirmation by clinicians.

Thereinafter, it will be understood that particular configurations, aspects, examples, clauses and embodiments described hereinafter are shown by way of illustration and not as limitations of the invention. The principal features of this invention can be employed in various embodiments without departing from the scope of the invention. Thus, one skilled in the art can easily ascertain the essential characteristics of the present invention and, without departing from the spirit and scope thereof, can make various changes and modifications of the invention to adapt it to various usages and conditions.

### Example 1

### 1. Virus Strain

A dengue virus serotype 1 (DENV 1, Taiwan virus strain 8700828), serotype 2 (DENV 2, virus strain 16681 and Taiwan virus strain 454009 A), serotype 3 (DENV 3, Taiwan virus strain 8700829), and serotype 4 (DENV 4, Taiwan virus strain 59201818) could be replicated in C6/36 cells using a conventional culture method. Virus culture was known to those of ordinary skill in the art of the present invention, so the details were not described herein. By using a commercially available centrifugation apparatus (for example, Macrosep® Advance Centrifugal Devices with a molecular weight cut-off of 30 kDa, Pall Corp., Port Washington, NY), the supernatant after removal of cells was concentrated into DE NV with a high viral titer at a rotation speed of 6000×g at 4° C., and then the DENV was stored in an environment lower than −70° C. for later use.

### 2. Serum Collection

This example used sera from 67 confirmed dengue patients, obtained in an acute phase (0-7 days after the onset of the disease) of these patients by the National Cheng Kung University Hospital (NCKUH) during the DENV outbreak in Tainan, Taiwan in 2015. The above sera were detected for dengue virus infection according to the laboratory standards established by the Taiwan Department of Disease Control. According to the latest principles published by the WHO, dengue fever patients could be grouped into severe patients, mild patients who had warning signs, and mild patients who had no warning signs according to the severity of the disease. In addition, this example used sera of 26 healthy volunteers as a negative control group. The collection of all the sera was carried out in accordance with the relevant criteria and regulations (IRB #A-BR-101-140) approved by the Institutional Review Board (IRB) of the NCKUH, and the informed consent of all participants and/or their legal representatives was obtained. 3. Sandwich ELISA

For sandwich ELISA, 100 μl mouse anti-human antibody against NS1 (the concentration is 2 μg/mL in PBS pH 7.2) was coated in a 96-well ELISA culture plate, and the plate was then placed at 4° C. overnight. After blocking with 200 μl PBS containing 1% BSA for 1 hour, 50 μl of patient serum (diluted at 1:5) or 50 μl of NS1 with known concentrations was cultured in the wells at 37° C. for 1 hour with a mouse anti-human antibody conjugated with biotin. After the wells were washed with PBS, 100 μl of streptavidin conjugated with horseradish peroxidase (HRP) diluted at 1:200 with PBS containing 1% BSA was added and incubated at 37° C. for 20 minutes. After the wells were washed with PBS, the TMB was used as the substrate for the chromogenic reaction. Subsequently, a termination solution (2N H₂SO₄) was added to the wells to stop the reaction, and the commercially available microplate reader was used to read the absorbance at OD450 nm. The concentration was obtained by comparing the absorbance at OD450 nm with a standard curve obtained by the absorbance at OD450 nm of the NS1 of known concentrations. The concentration of NS1 in the patient's serum corresponded to the first test result.

### 4. Indirect ELISA

For indirect ELISA, 50 μl NS1, bovine serum albumin (BSA), and peptides-conjugated BSA or an antibody (2 μg/ml, dissolved in PBS with pH 7.3) were separately coated in a 96-well E LISA culture plate, and the plate was then placed at 4° C. overnight. After blocking with PBS containing 1% BSA for 1 hour, the antibody [namely, the anti-NS1-WD peptide hAb, the hAb against NS1 of all serotypes (recognizing the 114^{th} to 119^{th} amino acids of the NS1), the anti-NS1-WD peptide mouse antibody (a monoclonal antibody strain 33D2; refer to SCIENTIFIC REPORTs 7: 6975, DOI:10.1038/s41598-017-07308-3, which was incorporated herein by reference), or the mouse antibody against NS1 of all serotypes (a monoclonal antibody strain 19-5, recognizing the 114^{th} to 119^{th} amino acid residues of the NS1, with; refer to SCIENTIFIC REPORTs 7: 6975, which was incorporated herein by reference)] with known concentrations or the patient serum (diluted at 1:50) was cultured in the wells and the reaction lasted for 1 hour at 37° C. The heavy chain variable regions CDRs and the light chain variable regions CDRs of the anti-NS1-WD peptide hAb and the hAb against NS1 of all serotypes were identical to those of the corresponding mouse monoclonal antibody strain above, but the remaining sequences were replaced with the sequences of the hAb. Results of the test using the anti-NS1-WD peptide mouse antibody and the mouse antibody against NS1 of all serotypes served as the comparative example. The sequences of the hAb other than the CDR sequences were known to those of ordinary skill in the art of the present invention, so the details were not described herein. Afterward, the anti-human IgG (Jackson ImmunoResearch Laboratories, West Grove, PA) secondary antibody (diluted at 1:10000) of conjugated horseradish peroxidase (HRP) was added to the wells, and the reaction lasted for another 1 hour at 37° C. After the wells were washed with PBST, tetramethylbenzidine (TMB; Clinical Science Products, Mansfield, MA) was used as the substrate for the chromogenic reaction. Subsequently, a termination solution (2N H₂SO₄) was added to the wells to stop reaction, and a commercially available microplate reader (for example, the VersaMax microplate reader; Molecular Devices, Sunnyvale, CA) was used to read the absorbance at OD450 nm. The concentrations of anti-NS1-WD peptide hAb and hAb against NS1 were obtained by comparing the absorbance at OD450 nm with standard curves obtained by the absorbance at OD450 nm of the anti-NS1-WD peptide hAb and hAb against NS1 of known concentrations, respectively. The content ratio of anti-NS1-WD peptide hAb and hAb against NS1 in the patient serum corresponded to the second test result.

### 5. Statistical Analysis

All numerical values were analyzed with Prism software (GraphPad, San Diego, CA). All the results were analyzed by means of unpaired Student's t-test or one-way ANOVA, so as to compare two or more independent groups. All the numerical values were obtained from three independent tests and expressed as mean±SD. In the statistical significance settings, the symbol * represented p<0.05, the symbol ** represented p<0.01, and the symbol *** represented p<0.001; and ns represented no significant difference in a 95% two-tailed confidence interval.

### Example 2

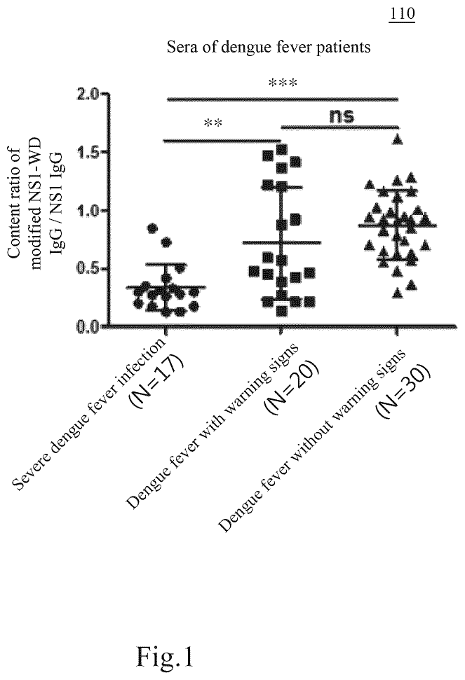

Following the method of example 1, an optical density (OD) value of the antibody (IgG) against modified NS1-WD peptide and an OD value of the antibody (IgG) against NS1 of all serotypes in the sera of the patients were separately detected, and then a ratio (NS1-WD IgG/NS1 IgG) of the OD values was used to evaluate the dengue fever patients varying in severity. The result was shown in FIG. 1.

Referring to FIG. 1, FIG. 1 showed a scatter plot 110 illustrating a content ratio of anti-modified NS1-WD IgG/anti-NS1 IgG in sera of various dengue fever patients according to an example of the present invention, where the symbol * represented p<0.05, the symbol ** represented p<0.01, and the symbol *** represented p<0.001.

As shown in FIG. 1, compared to the sera (N=20) of the dengue fever patients having warning signs or the sera (N=30) of the dengue fever patients having no warning signs, the sera (N=17) of the patients with severe dengue had a significantly reduced content ratio of anti-NS1-WD IgG/anti-NS1 IgG. After further analysis, there was no statistically significant difference between the OD values of anti-NS1 IgG of all the patients, which indicated that the test of the anti-NS1-WD peptide antibody indeed facilitated improvement of the prediction accuracy of the dengue fever patients.

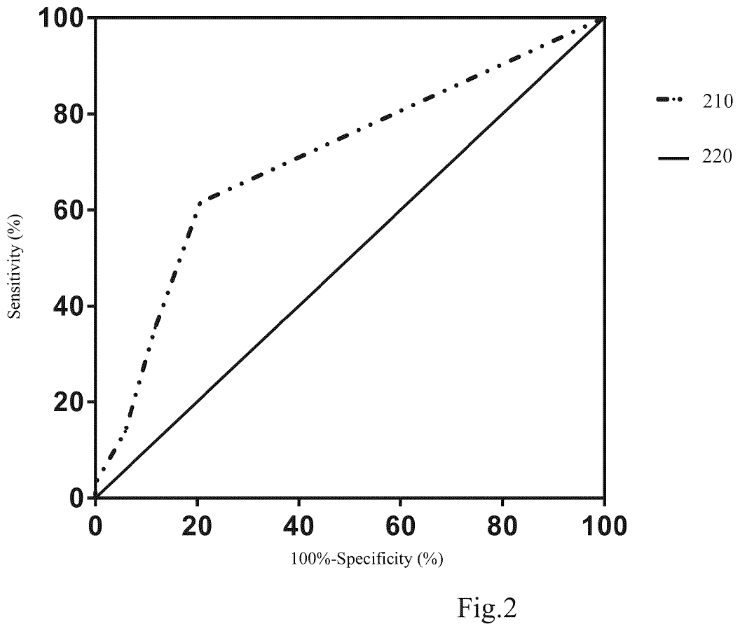

Referring to FIG. 2, FIG. 2 showed a ROC curve in severe dengue prediction with a conventional method, wherein curve 210 represented the ROC curve and curve 220 represented an identity line. The conventional method used in FIG. 2 included analyzing the symptoms, a medical history, and a simple non-specific test result (including a single test result or a test result without targeting anti-NS1-WD IgG) of the patients.

It could be seen from the result of FIG. 2 that each point of the ROC curve in severe dengue prediction with the conventional test method (only based on the symptoms, medical history, and simple non-specific test result) lay above curve 220, and the area under curve (AUC) was 0.7036 (with a confidence interval of 0.60 to 0.81).

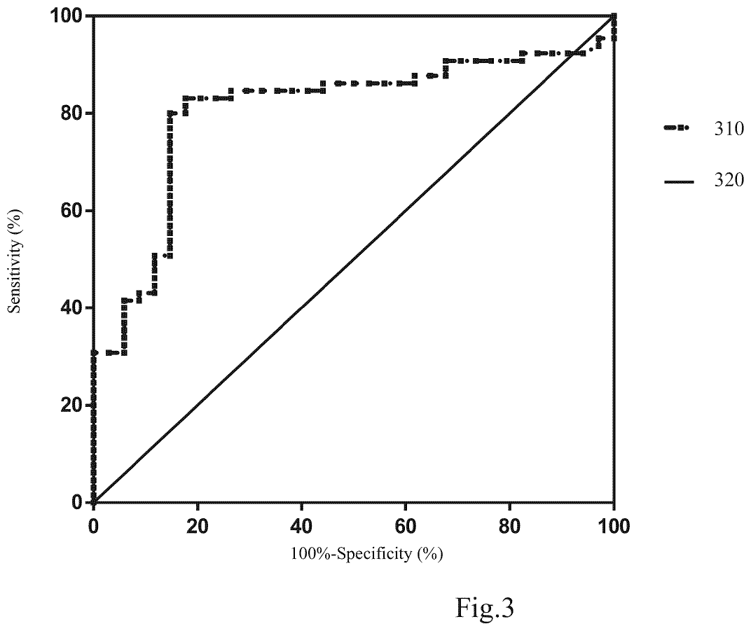

Referring to FIG. 3, FIG. 3 showed a ROC curve in a severe dengue prediction in terms of NS1 antigens in the sera of dengue fever patients according to an example of the present invention, in which the x-axis represented sensitivity, the y-axis represented an opposite of specificity (i.e., 100%-specificity) at each cutoff value, curve 310 represented the ROC curve, and curve 320 represented an identity line. It could be seen from FIG. 3 that although each single point on the ROC curve of the severe dengue prediction in terms of the NS1 antigens in the sera of dengue fever patients lay above curve 310, the single point on the ROC curve with higher sensitivity (about 90%) lay below curve 320. The AUC was 0.8052 (with a confidence interval of 0.71 to 0.90), the sensitivity was 80.00% and the specificity was 77.14% when the cut-off value of the OD value was taken as 0.8040, which can be converted to a concentration of 666.27 ng/ml.

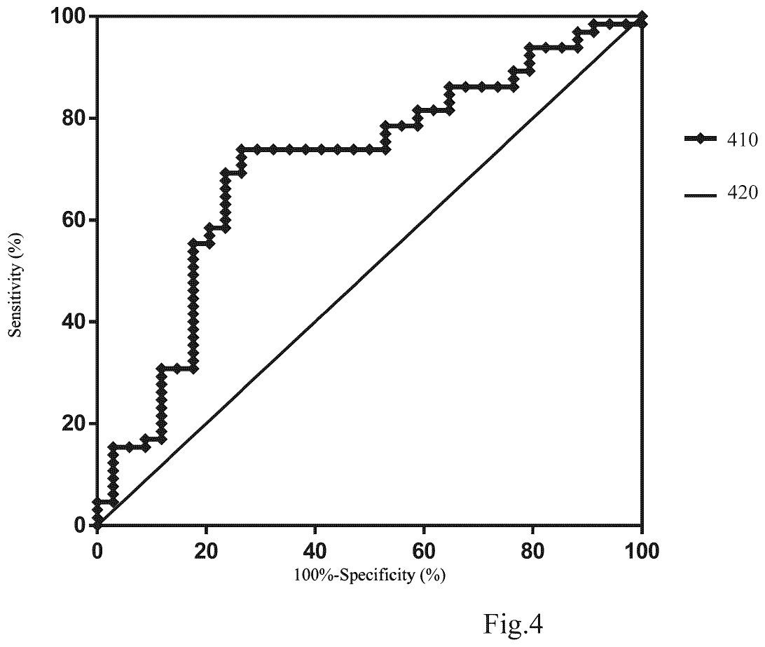

Referring to FIG. 4, FIG. 4 showed a ROC curve in a severe dengue prediction in terms of a content ratio of the endogenous anti-NS1 antibodies (i.e., anti-NS1-WD peptide antibody/anti-NS1 peptide antibody) in the sera of dengue fever patients according to an example of the present invention, in which the x-axis represented sensitivity, the y-axis represented an opposite of specificity (i.e., 100%-specificity) at each cutoff value, curve 410 represented the ROC curve, and curve 420 represented an identity line. It could be seen from the result of FIG. 4 that each single point on the ROC curve of the severe dengue prediction in terms of the endogenous anti-NS1 antibody in the sera of the dengue fever patients lay above curve 420, where the AUC was 0.7027 (with a confidence interval of 0.59 to 0.81), the sensitivity was 72.31% and the specificity was 76.47% when the cut-off value of OD value was taken as 0.4294.

Referring to TABLE 1, TABLE 1 showed the number of patients grouped by detecting and crossly comparing the NS1 antigen and the content ratio of the endogenous anti-NS1 antibody (i.e., anti-NS1-WD peptide antibody/anti-NS1 peptide antibody) in the sera of dengue fever patients according to an example of the present invention, and a result confirmed after differential diagnosis, where the severe and mild diseases were confirmed by post differential diagnosis for the subjects according to Handbook for Clinical Management of Dengue published by the WHO.

TABLE 1

| | Severe | Mild |
|---|---|---|
| 99 in total | (65) | (34) |
| NS1 antigen was higher than 666.27 ng/ml or content | 62 | 13 |
| ratio of the endogenous was lower than 0.4294 | | |
| NS1 antigen was not higher than 666.27 ng/ml and | 3 | 21 |
| content ratio of the endogenous was lower than 0.4294 | | |

The content ratio of the endogenous anti-NS1 antibody was detected by using the hAb. As shown in TABLE 1, when the sera of the dengue fever patients had concentrations of NS1 antigen higher than 666.27 ng/ml, or had a content ratio of endogenous anti-NS1 of lower than 0.4294, the subject corresponding to the ex vivo biological specimen was predicted as the severe dengue. After confirmation through post-diagnosis or differential diagnosis, the true positive rate (also referred to as prediction accuracy) of severe dengue prediction by using the method of the present invention could reach up to 95.38% (namely, 62/65=95.38%), that is, the false negative rate (namely, the value of P) was reduced to 4.62%. Therefore, the method of the present invention was applicable to predict the severe dengue in a subject, and could be used as a reference for clinical staff to assess the risk and/or make treatment strategies. It was noted that when the dengue fever in a subject was predicted by only the concentration of NS1 antigen or the content ratio of endogenous anti-NS1 antibody (i.e., anti-NS1-WD peptide antibody/anti-NS1 peptide antibody) in the sera, the false negative rate (i.e., opposite of sensitivity) was 22.86% (100%-77.14%) and 23.53% (100%-76.47%), respectively. However, by predicting the dengue fever patient to be severe dengue or mild dengue with both the concentration of NS1 antigen and the content ratio of endogenous anti-NS1 antibody (i.e., anti-NS1-WD peptide antibody/anti-NS1 peptide antibody) in the sera, the false negative rate was much lower (4.62%). That is, by detecting both the concentration of NS1 antigen and the content ratio of endogenous anti-NS1 antibodies (i.e., anti-NS1-WD peptide antibody/anti-NS1 peptide antibody) in the sera of a dengue fever patient, the false negative rate to predict a subject with severe dengue as a subject with non-severe dengue becomes lower.

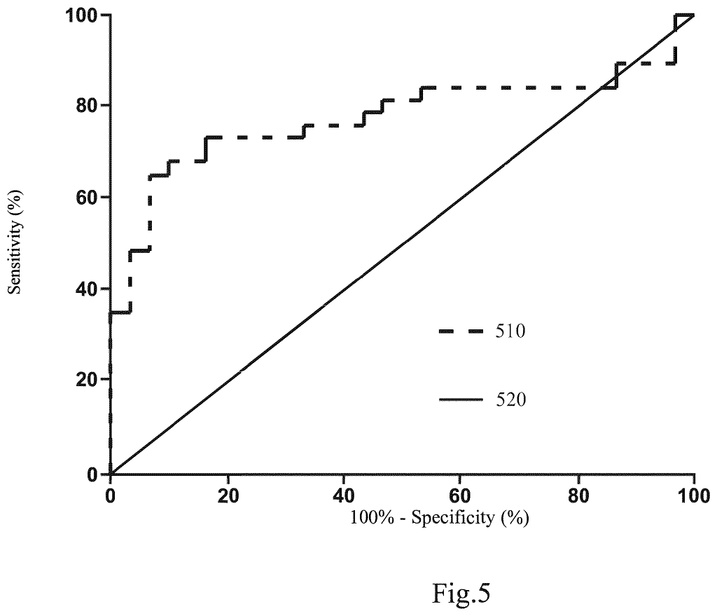

In comparison, if severe dengue in a subject was predicted by detecting the content ratio of the endogenous anti-NS1 antibodies with a mouse antibody, the resulting false negative rate was high. Referring to FIG. 5, FIG. 5 showed a ROC curve in a severe dengue prediction in terms of a content ratio of the endogenous anti-NS1 antibodies in the sera of dengue fever patients by means of mouse antibody detection according to a comparative example, in which the x-axis represented sensitivity, the y-axis represented an opposite of specificity (i.e., 100%-specificity) at each cutoff value, curve 510 represented the ROC curve, and curve 520 represented chance. It could be seen from the result of FIG. 5 that although the AUC was 0.7739 (with a confidence interval of 0.6557 to 0.8921), the single point on the ROC curve with higher sensitivity (about 90%) lay below curve 520.

Referring to TABLE 2, TABLE 2 showed the number of severe and mild dengue patients obtained by detecting the content ratio of the endogenous anti-NS1 antibodies in the sera of dengue fever patients with the mouse antibody according to the comparative example, and a result confirmed after differential diagnosis, where the severe and mild diseases were confirmed by post differential diagnosis for the subjects according to Handbook for Clinical Management of Dengue published by the WHO.

TABLE 2

| | Severe | Mild |
|---|---|---|
| 67 in total | (37) | (30) |
| Content ratio of Anti-NS1 antibody | 27 | 5 |
| lower than 0.4294 | | |
| Content ratio of Anti-NS1 antibody | 10 | 25 |
| not lower than 0.4294 | | |

It could be seen from TABLE 2 that, when the endogenous anti-NS1 antibody in the sera of the dengue fever patients was detected to be lower than 0.4294 with the mouse antibody, it was determined that the subject corresponding to the ex vivo biological specimen was predicted as developing severe dengue. After confirmation through post-diagnosis or differential diagnosis, the true positive rate (also referred to as prediction accuracy) of predicting subjects with severe dengue by the method in the comparative example was merely 72.9% (namely, 27/37=72.9%), and the false negative rate reached up to 27.1% (namely, 10/37=27.1%), which was indeed much higher than the false negative rate (4.5%) of the method of the present invention.

To sum up, the above-described specific antigen, specific antibody, specific patient group, specific analysis mode, or specific evaluation method is merely used to illustrate the method of improving prediction accuracy of severe dengue prediction in a subject. However, those of ordinary skill in the art of the present invention should understand that other antigens, other antibodies, other patient groups, other analysis modes, or other evaluation methods can also be used in the method of elevating prediction accuracy of severe dengue prediction without departing from the spirit and scope of the present invention, so the present invention is not limited to the above description. For example, without negatively affecting the true positive rate or raising false negative rate, the first test result can be obtained by detecting the NS1 and/or NS1 complex in other manners or the second test result can be obtained by detecting the endogenous anti-NS1 antibodies in other manners.

It can be seen from the aforementioned embodiments that, in the method of improving the prediction accuracy of severe dengue prediction in a subject, a non-structural protein 1 (NS1) and an endogenous anti-NS1 antibody of dengue virus in an ex vivo biological specimen can be detected and crossly compared, leading in reduction of false negative rates of testing results as well as improved prediction accuracy of severe dengue prediction in a subject.

Although the present invention has been disclosed above with several specific examples, various modifications, changes and substitutions can be made to the foregoing disclosure. Moreover, it should be understood that, without departing from the spirit and scope of the present invention, certain features of the examples of the present invention will be used in some cases, but other features are not used correspondingly. Therefore, the spirit and the scope of the claims of the present invention should not be limited to those described in the above exemplary examples.

What is claimed is:

1. A method of improving prediction accuracy of severe dengue prediction in a subject, comprising:
providing an ex vivo biological specimen, wherein the ex vivo biological specimen has not been diagnosed or differentially diagnosed with a dengue virus infection or a suspected dengue virus infection;
performing two detection steps on the ex vivo biological specimen, for obtaining a first test result and a second test result, wherein the first test result comprises a concentration of non-structural protein 1 (NS1), and the second test result comprises a content ratio of a first antibody and a second antibody, the first antibody specifically recognizes the 109^{th} to the 122^{nd} amino acid residues of the NS1, and the second antibody specifically recognizes the 114^{th} to the 119^{th} amino acid residues; and
diagnosing the subject with severe dengue when the first test result is higher than a first cut-off value and the second test result is lower than a second cut-off value, wherein a false negative rate of the severe dengue is lower than 5%.

2. The method of claim 1, wherein the ex vivo biological specimen comprises blood, urine, saliva, tissue fluid and/or lymphatic fluid.

3. The method of claim 1, wherein the first cut-off value is 660 ng/ml to 670 ng/ml.

4. The method of claim 1, wherein the second cut-off value is 0.4294.

5. The method of claim 1, wherein a serotype of the dengue virus is selected from the group consisting of type 1, type 2, type 3 and type 4.

6. The method of claim 1, wherein an isotype of the first antibody and the second antibody are IgG and/or IgM.

7. The method of claim 1, wherein the subject is a mammal.

8. The method of claim 1, wherein the subject is a human being.

9. The method of claim 1, wherein a true positive rate of the severe dengue prediction is at least 95%.

10. The method of claim 1, wherein the two detection steps are performed by immunoassays comprising enzyme-linked immunosorbent assay (ELISA), western blot assay, lateral laminar flow immunoassay, multiple immunoassay, radio immunoassay, immunoradiometric assay (IRMA), fluorescence immunoassay (FIA), chemiluminescence immunoassay (CLIA) and/or immunoturbidimetry.

11. A method of reducing a false negative rate of a severe dengue prediction in a subject, comprising:
providing an ex vivo biological specimen, wherein the ex vivo biological specimen has not been diagnosed or differentially diagnosed with a dengue virus infection or a suspected dengue virus infection;
performing two detection steps on the ex vivo biological specimen, for obtaining a first test result and a second test result, wherein the first test result comprises a concentration of non-structural protein 1 (NS1), and the second test result comprises a content ratio of a first antibody and a second antibody, the first antibody specifically recognizes the 109^{th} to the 122^{nd} amino acid residues of the NS1, the second antibody specifically recognizes the 114^{th} to the 119^{th} amino acid residues; and
diagnosing the subject with severe dengue when the first test result is higher than 650 ng/mL to 670 ng/mL or the second test result is lower than 0.40 to 0.45, wherein a false negative rate of the severe dengue prediction is lower than 5%.

12. The method of claim 11, wherein the ex vivo biological specimen comprises blood, urine, saliva, tissue fluid and/or lymphatic fluid.

13. The method of claim 11, wherein a serotype of the dengue virus comprises type 1, type 2, type 3 and type 4.

14. The method of claim 11, wherein an isotype of the first antibody and a second antibody are IgG and/or IgM.

15. The method of claim 11, wherein the subject is a mammal.

16. The method of claim 11, wherein the subject is a human being.

17. The method of claim 11, wherein a true positive rate of the severe dengue prediction is at least 95%.

18. The method of claim 11, wherein the two detection steps are performed by immunoassays comprising enzyme-linked immunosorbent assay (ELISA), western blot assay, lateral laminar flow immunoassay, multiple immunoassay, radio immunoassay, immunoradiometric assay (IRMA), fluorescence immunoassay (FIA), chemiluminescence immunoassay (CLIA) and/or immunoturbidimetry.
